(19) United States
(12) Patent Application Publication
(10) Pub. No.: US 2026/0224364 A1
(43) Pub. Date: Aug. 6, 2026

(54) STABILIZATION AND CONTROL MECHANISM FOR PLACEMENT OF LEAFLET CUTTING DEVICE

(71) Applicant: BOSTON SCIENTIFIC SCIMED, INC., MAPLE GROVE, MN (US)

(72) Inventors: Daniel Presteng, Corcoran, MN (US); Joseph Edward Adriaens, Elk River, MN (US); Philip Andrew Litecky, Forest Lake, MN (US); Eric Michael Petersen, Maple Grove, MN (US); Joel T. Eggert, Plymouth, MN (US)

(73) Assignee: BOSTON SCIENTIFIC SCIMED, INC., MAPLE GROVE, MN (US)

(21) Appl. No.: 19/529,690

(22) Filed: Feb. 4, 2026

Related U.S. Application Data
(60) Provisional application No. 63/753,592, filed on Feb. 4, 2025.

(51) Int. Cl. A61F 2/24; A61B 17/3207

(52) U.S. Cl. CPC A61F 2/2445; A61B 17/320725; A61F 2/2418; A61F 2/2436; A61F 2/2442; A61F 2/2466

(57) ABSTRACT

A medical device for positioning a cutting device relative to a heart valve leaflet includes an elongate shaft extending from a proximal region to a distal region and configured to accept a cutting device therethrough. A stabilization loop extends from the distal region of the shaft, featuring first and second loop sides that terminate in respective ends. The device includes a control knob slidably mounted on the elongate shaft and coupled to the stabilization loop. The control knob is configured to translate along the shaft to move the stabilization loop and can be rotated to selectively lengthen one of the first or second loop sides, thereby moving the distal region relative to the heart valve. This selective lengthening capability enables precise positioning of the device relative to the valve leaflet during cardiac procedures such as valve leaflet laceration.

### CROSS REFERENCE TO RELATED APPLICATIONS

This application claims the benefit of priority of U.S. Provisional Application No. 63/753,592 filed February 4, 2025, the entire disclosure of which is hereby incorporated by reference.

### TECHNICAL FIELD

The present disclosure relates generally to medical devices. More particularly, the present disclosure pertains to medical devices for excising cardiac valve leaflets.

### BACKGROUND

A wide variety of intracorporeal medical devices have been developed for medical use, and more specifically for intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for excising cardiac valve leaflets. An example may be found in a medical device that is configured for positioning a cutting device relative to a valve leaflet of a heart valve. The medical device includes an elongate shaft extending from a proximal region to a distal region and is configured to accept the cutting device extending therethrough. The device has a stabilization loop extending out of the distal region of the elongate shaft, where the stabilization loop includes a first loop side terminating in a first end and a second loop side terminating in a second end. A control knob is slidingly disposed relative to the elongate shaft and is adapted to slide relative to the elongate shaft to translate the stabilization loop. The control knob engages with the stabilization loop such that rotating the control knob selectively lengthens one of the first loop side and the second loop side, thereby moving the distal region of the elongate shaft relative to the heart valve.

Alternatively or additionally, rotating the control knob may selectively lengthen one of the first loop side and the second loop side and shorten the other of the first loop side and the second loop side, thereby moving the distal region of the elongate shaft relative to the heart valve.

Alternatively or additionally, the first end may be secured to a first side of the control knob and the second end may be secured to a second side of the control knob.

Alternatively or additionally, translating the control knob distally relative to the elongate shaft might cause the stabilization loop to move distally.

Alternatively or additionally, the elongate shaft may contain a plurality of lumens.

Alternatively or additionally, the plurality of lumens might include a first stabilization loop lumen accommodating the first loop side extending therethrough and a second stabilization loop lumen accommodating the second loop side extending therethrough.

Alternatively or additionally, the plurality of lumens may include a guidewire lumen.

Alternatively or additionally, the plurality of lumens might include a cutting device lumen.

Alternatively or additionally, the plurality of lumens may include an access lumen adapted to accommodate a guidewire and/or a cutting device.

Alternatively or additionally, the stabilization loop may include a stainless steel wire.

Alternatively or additionally, the stabilization loop may include a nitinol wire.

Another example is found in a medical device that is adapted for positioning a cutting device relative to a valve leaflet of a heart valve. The medical device includes an elongate shaft having a plurality of lumens extending from a proximal region to a distal region. The device has a stabilization loop extending out of the distal region of the elongate shaft. The stabilization loop includes a first loop side extending through a first lumen of the plurality of lumens and a second loop side extending through a second lumen of the plurality of lumens. The first loop side terminates in a first end and the second loop side terminates in a second end. A control knob is disposed relative to the elongate shaft and is engaged with the stabilization loop such that rotating the control knob selectively lengthens one of the first loop side and the second loop side.

Alternatively or additionally, the first end may be coupled with a first side of the control knob and the second end may be coupled with a second side of the control knob.

Alternatively or additionally, the device may include an elongate slot formed within the elongate shaft, where the elongate slot might allow the control knob to translate relative to the elongate shaft, thereby causing the stabilization loop to translate relative to the elongate shaft.

Alternatively or additionally, rotating the control knob may selectively lengthen one of the first loop side and the second loop side and shorten the other of the first loop side and the second loop side, thereby moving the distal region of the elongate shaft relative to the heart valve.

Alternatively or additionally, the plurality of lumens may include a guidewire lumen.

Alternatively or additionally, the plurality of lumens may include a cutting device lumen.

Alternatively or additionally, the stabilization loop may be made of stainless steel wire.

Alternatively or additionally, the stabilization loop may be made of nitinol wire.

Another example is found in a medical device adapted for positioning a cutting device relative to a valve leaflet of a heart valve. The medical device includes an elongate shaft having a plurality of lumens extending through the elongate shaft, where the elongate shaft extends from a proximal region to a distal region. The device has a stabilization loop extending out of the distal region of the elongate shaft, where the stabilization loop includes a first end extending proximally through a first lumen of the plurality of lumens and a second end extending proximally through a second lumen of the plurality of lumens. A control knob is slidingly disposed relative to the elongate shaft. The first end of the stabilization loop is secured to a first side of the control knob and the second end of the stabilization loop is secured to a second side of the control knob. The control knob is adapted to slide relative to the elongate shaft to translate the stabilization loop distally and proximally, and the control knob is adapted to advance a first side of the stabilization loop in response to rotating the control knob a first rotational direction and to advance a second side of the stabilization loop in response to rotating the control knob a second rotational direction.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing an aortic heart valve positioned within a native valve annulus of a heart;

FIGS. 2A and 2B are partial cutaway views of a native valve anulus of a heart;

FIG. 3 is a partial cutaway view showing an illustrative medical device disposed proximate the native valve annulus shown in FIG. 2B, with a stabilization loop of the medical device shown in a retracted position;

FIG. 4 is a partial cutaway view showing the illustrative medical device of FIG. 3 with the stabilization loop of the medical device shown in an extended position;

FIG. 5 is a partial cutaway view showing the illustrative medical device of FIG. 3, with the stabilization loop further extended in order to better position the medical device relative to the coronary ostium; and

FIG. 6 is a cross-sectional view taken along the line 6-6 of FIG. 3.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove or excise one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine pericardium, or may be polymeric. In some instances, artificial valve leaflets may be made of polymers such as Dacron or Gore-Tex. As discussed here, reference to a valve leaflet may refer to either a native valve leaflet or an artificial valve leaflet.

In some instances, a medical device may be adapted for positioning a leaflet cutting device relative to a valve leaflet of a heart valve. The medical device includes an elongate shaft that extends from a proximal region to a distal region and is configured to accept the cutting device extending therethrough. A stabilization loop extends out of the distal region of the elongate shaft and that includes a first loop side terminating in a first end and a second loop side terminating in a second end. A control knob is slidingly disposed relative to the elongate shaft and is adapted to slide relative to the elongate shaft in order to translate the stabilization loop. The control knob is engaged with the stabilization loop such that rotating the control knob selectively lengthens one of the first loop side and the second loop side, thereby moving the distal region of the elongate shaft relative to the heart valve.

In some cases, rotating the control knob may selectively lengthen one of the first loop side and the second loop side and may shorten the other of the first loop side and the second loop side, thereby moving the distal region of the elongate shaft relative to the heart valve. In some cases, the first end may be secured to a first side of the control knob and the second end may be secured to a second side of the control knob. Translating the control knob distally relative to the elongate shaft may cause the stabilization loop to move distally. In some cases, the elongate shaft may include a plurality of lumens. The plurality of lumens may include a first stabilization loop lumen that accommodates the first loop side extending therethrough and a second stabilization loop lumen that accommodates the second loop side extending therethrough. In some cases, the plurality of lumens may include a guidewire lumen. In some cases, the plurality of lumens may include a cutting device lumen. The plurality of lumens may include an access lumen that is adapted to accommodate a guidewire and/or a leaflet cutting device. In some cases, the stabilization loop may include a stainless steel wire. In some cases, the stabilization loop may include a nitinol wire.

In some instances, a medical device may be adapted for positioning a leaflet cutting device relative to a valve leaflet of a heart valve. The medical device includes an elongate shaft including a plurality of lumens extending from a proximal region to a distal region. A stabilization loop extends out of the distal region of the elongate shaft and includes a first loop side extending through a first lumen of the plurality of lumens and a second loop side extending through a second lumen of the plurality of lumens. The first loop side terminates in a first end and the second loop side terminates in a second end. A control knob is disposed relative to the elongate shaft and is engaged with the stabilization loop such that rotating the control knob selectively lengthens one of the first loop side and the second loop side.

In some cases, the first end may be coupled with a first side of the control knob and the second end may be coupled with a second side of the control knob. The medical device may further include an elongate slot that is formed within the elongate shaft. The elongate slot may allow the control knob to translate relative to the elongate shaft, thereby causing the stabilization loop to translate relative to the elongate shaft. In some cases, rotating the control knob may selectively lengthen one of the first loop side and the second loop side and may shorten the other of the first loop side and the second loop side, thereby moving the distal region of the elongate shaft relative to the heart valve. In some cases, the plurality of lumens includes a guidewire lumen. In some cases, the plurality of lumens may include a leaflet cutting device lumen. In some cases, the stabilization loop may include a stainless steel wire. In some cases, the stabilization loop may include a nitinol wire.

In some instances, a medical device may be adapted for positioning a leaflet cutting device relative to a valve leaflet of a heart valve. The medical device includes an elongate shaft that defines a plurality of lumens extending through the elongate shaft. The elongate shaft extends from a proximal region to a distal region. A stabilization loop extends out of the distal region of the elongate shaft and includes a first end extending proximally through a first lumen of the plurality of lumens and a second end extending proximally through a second lumen of the plurality of lumens. A control knob is slidingly disposed relative to the elongate shaft, with the first end of the stabilization loop secured to a first side of the control knob and the second end of the stabilization loop secured to a second side of the control knob. The control knob is adapted to slide relative to the elongate shaft in order to translate the stabilization loop distally and proximally. The control knob is adapted to advance a first side of the stabilization loop in response to rotating the control knob a first rotational direction and is adapted to advance a second side of the stabilization loop in response to rotating the control knob a second rotational direction.

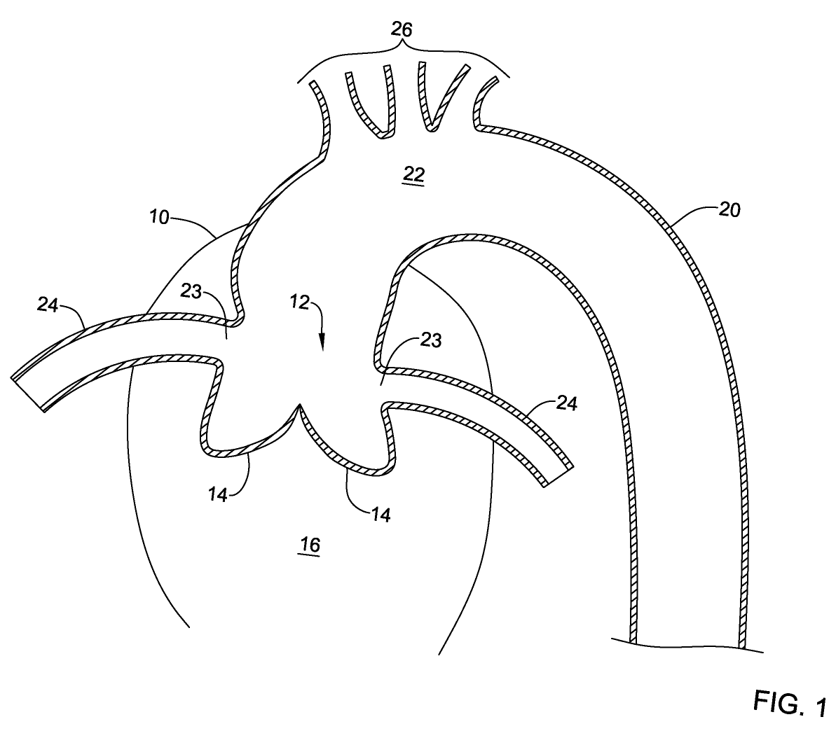

FIG. 1 is a schematic partial cut-away view of a portion of a patient’s heart 10 including an aortic valve 12 having native valve leaflets 14 disposed within and/or extending from a native valve annulus, a left ventricle 16, and certain connected vasculature, such as an aorta 20 connected to the aortic valve 12 of the patient’s heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. While the aortic valve 12 includes a total of three native valve leaflets 14, only two are visible in the illustrated cutaway view. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

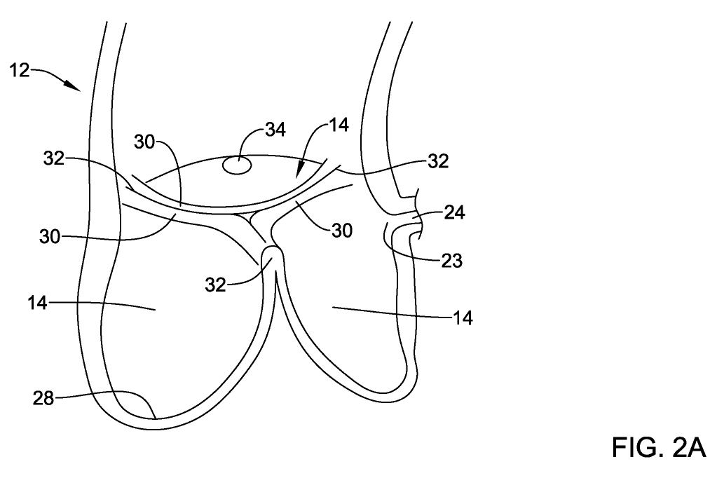

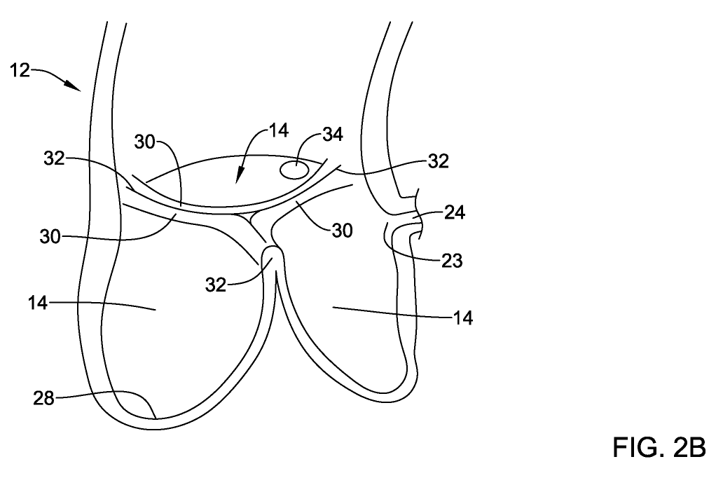

FIGS. 2A and 2B provide additional views of the aortic valve 12. As shown in this particular cutaway of the aortic valve 12, all three of the native valve leaflets 14 are visible. Each of the native valve leaflets 14 may be considered as extending from a leaflet nadir 28 to a leaflet top 30. Each of the leaflet tops 30 come together when the aortic valve 12 is in a closed configuration (as shown in FIGS. 2A and 2B), and move away from each other when the aortic valve 12 opens to let blood flow through the aortic valve 12 and into the aorta 20. The junction of two adjoining leaflet tops 30 may be considered as defining a commissure 32. A coronary ostium 23 leads to one of the coronary arteries 24. The relative locations of the coronary ostia 23 relative to the valve leaflets 14 can vary from patient to patient. In FIG. 2A, a coronary ostium 34 leading to an unseen coronary artery 24, is centered or roughly centered relative to one of the native valve leaflets 14. In FIG. 2B, the coronary ostium is off-center relative to one of the native valve leaflets 14. The coronary ostium 34 is not visible in the cutaway view of FIG. 1. In situations in which there is a desire to lacerate the native valve leaflet 14 in line with the coronary ostium 34, it can be beneficial to be able to direct a leaflet cutting device (not shown) to a position in which the leaflet cutting device is centered over the coronary ostium 34.

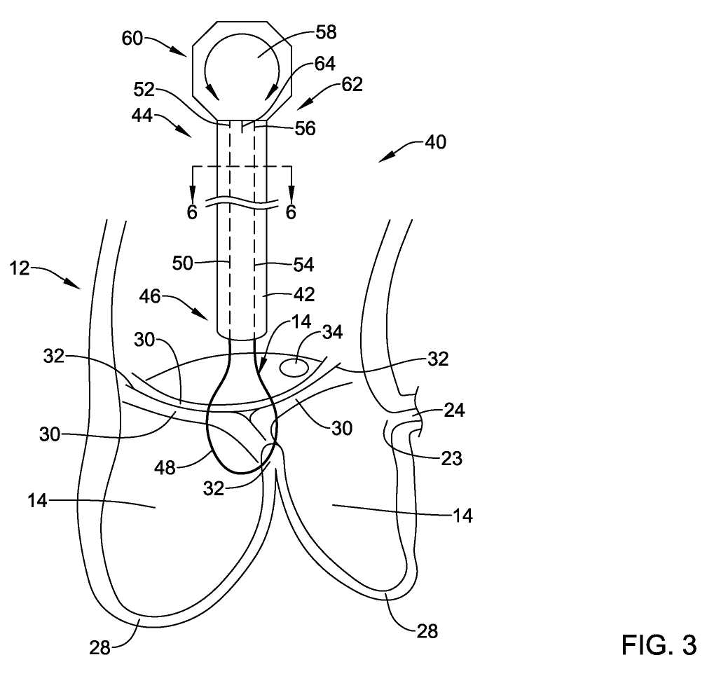

FIG. 3 is a partial cutaway view of the anatomy, showing an illustrative medical device 40 extending distally through the aorta 20 and into a position proximate the aortic valve 12. In FIG. 3, the coronary ostium 34 is shown as being off-centered relative to a valve leaflet 14. While the medical device 40 is shown with respect to positioning relative to an aortic valve, it will be appreciated that the medical device 40 may be used in a similar manner for relative positioning with any other cardiac valve as well. While the medical device 40 is shown with respect to positioning relative to a native aortic valve 12, the medical device 40 may similarly be used for positioning a leaflet cutting device relative to a previously implanted replacement heart valve. Some non-limiting examples of a replacement heart valves with which the medical device 40 may be utilized include the ACURATE NEO2™, the ACURATE PRIME™, and/or family members thereof from Boston Scientific.

The medical device 40 includes an elongate shaft 42 that extends from a proximal region 44 to a distal region 46. The elongate shaft 42 is adapted to accept a leaflet cutting device extending through the elongate shaft 42 once the distal region 46 of the elongate shaft 42 has been appropriately positioned relative to the coronary ostium 34. Any of a variety of different leaflet cutting devices may be used. Some leaflet cutting devices mechanically cut through valve leaflets. Some leaflet cutting devices utilize RF (Radio Frequency) energy and an appropriately shaped electrode through which the RF energy is applied to cut through valve leaflets.

The medical device 40 includes a stabilization loop 48 that extends out of the distal region 46 of the elongate shaft 42. In some cases, the stabilization loop 48 engages tissue within the aortic valve 12 to stabilize the relative position of the distal region 46 of the elongate shaft 42 prior to advancing a leaflet cutting device through the elongate shaft 42. In some cases, the stabilization loop 48 also helps to position the distal region 46 of the elongate shaft 42 relative to the anatomy, including the coronary ostium 34. The stabilization loop 48 includes a first loop side 50 (shown in phantom) that terminates in a first end 52 and a second loop side 54 (shown in phantom) that terminates in a second end 56. In some cases, the stabilization loop 48 may be a stainless steel wire. In some cases, the stabilization loop 48 may be a nitinol wire. A control knob 58 is slidingly disposed relative to the elongate shaft 42. In some cases, the control knob 58 is adapted to slide axially relative to the elongate shaft 42 in order to cause the stabilization loop 48 to translate.

In some cases, the control knob 58 is engaged with the stabilization loop 48 such that rotating the control knob selectively lengthens one of the first loop side 50 and the second loop side 54, thereby causing the stabilization loop 48 to engage with the anatomy and urge the distal region 46 of the elongate shaft 42 to one side or the other of the anatomy proximate the aortic valve 12. In some cases, rotating the control knob 58 selectively lengthens one of the first loop side 50 and the second loop side 54 and shortens the other of the first loop side 50 and the second loop side 54, thereby moving the distal region 46 of the elongate shaft 42 relative to the aortic valve 12. In some cases, the first end 52 of the first loop side 50 may be secured to a first side 60 of the control knob 58 and the second end 56 of the second loop 54 may be secured to a second side 62 of the control knob 58. In some cases, the elongate shaft 42 includes an elongate slot 64 that allows the control knob 58 to translate relative to the elongate shaft 42. Translating the control knob 58 in a distal direction causes the stabilization loop 48 to move distally, for example.

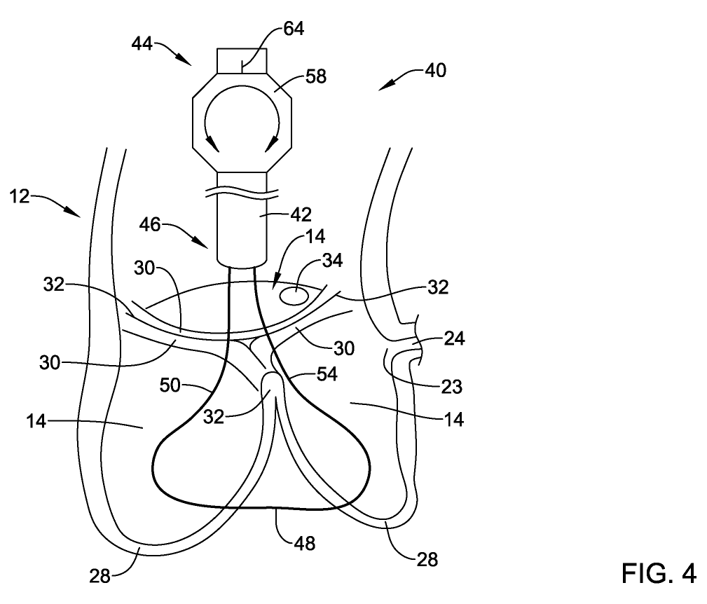

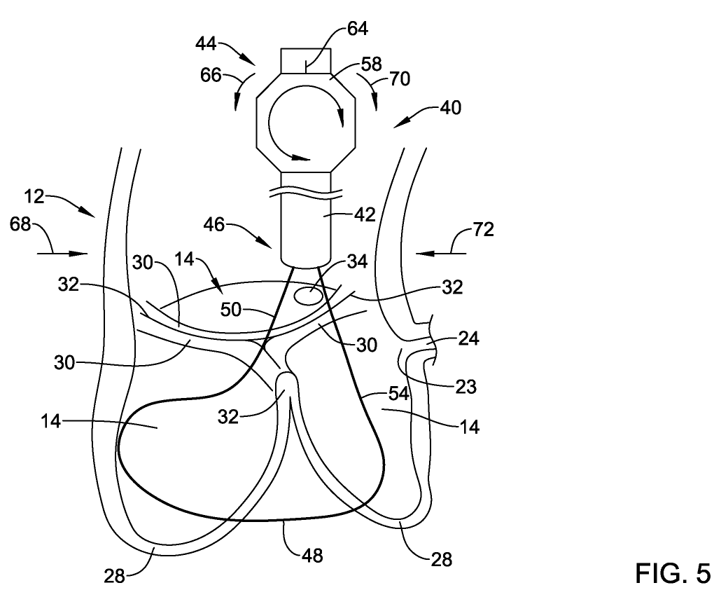

As shown in FIG. 3, the stabilization loop 48 is in a partially retracted configuration in which the stabilization loop 48 extends out of the distal region 46 of the elongate shaft 42 but has not yet reached the leaflet nadir 28. Moving to FIG. 4, the control knob 58 has been moved distally relative to the elongate shaft 42, thereby causing the stabilization loop 48 to extend further out of the distal region 46 of the elongate shaft 42. At this point, the distal region 46 of the elongate shaft 42 is roughly centered within the aortic valve 12 while the coronary ostium 34 is not centered. The distal region 46 of the elongate shaft 42 may be urged to the side, closer to being aligned with the coronary ostium 34, by appropriately rotating the control knob 58, as shown in FIG. 5.

In FIG. 5, the control knob 58 has been rotated in a direction indicated by an arrow 66. As a result, the first loop side 50 of the stabilization loop 48 has lengthened. Lengthening the first side loop 50 of the stabilization loop 48 urges the distal region 46 of the elongate shaft 42 to move in a direction indicated by an arrow 68. In some cases, rotating the control knob 58 in the direction indicated by the arrow 66 also results in the second loop side 54 of the stabilization loop 48 being shortened. In some cases, rotating the control knob 58 in a direction indicated by an arrow 70 causes the second loop side 54 of the stabilization loop 48 to be lengthened. Lengthening the second loop side 54 of the stabilization loop 48 will cause the stabilization loop 48 to urge the distal region 46 of the elongate shaft 42 in a direction indicated by an arrow 72. In some cases, rotating the control knob 58 in the direction indicated by the arrow 70 also results in the first loop side 50 of the stabilization loop 48 to be shortened.

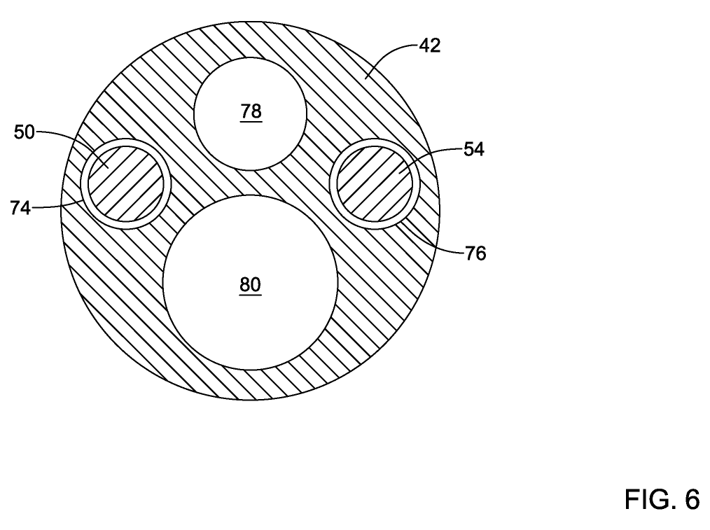

The elongate shaft 42 is adapted to allow the stabilization loop 48 to extend through the elongate shaft 42, extending distally from the control knob 48. In some cases, the elongate shaft 42 may be adapted to also accommodate a guidewire. A guidewire (not shown) may be advanced through the vasculature and may extend to or even through the aortic valve 12, and the medical device 40 may then be advanced over the guidewire. In some cases, the elongate shaft 42 may be adapted to also accommodate a leaflet cutting device (not shown). FIG. 6 is a cross-sectional view taken along the line 6-6 of FIG. 3, providing an example of an internal structure of the elongate shaft 42. As shown, the elongate shaft 42 may include a plurality of lumens. The plurality of lumens may include a first stabilization loop lumen 74 that accommodates the first loop side 50 of the stabilization loop 48 and a second stabilization loop lumen 76 that accommodates the second loop side 54 of the stabilization loop 48. The first stabilization loop lumen 74 and the second stabilization loop lumen 76 may have diameters that are dictated by the diameter of the wire used as the stabilization loop 48. The plurality of lumens may include a guidewire lumen 78. The guidewire lumen 78 may have a diameter that is dictated by the diameter of a guidewire expected to be used with the medical device 40. The plurality of lumens may include a leaflet cutting device lumen 80. As an example, the leaflet cutting device lumen 80 may have a 6 French to 8 French diameter.

The plurality of lumens, including the first stabilization loop lumen 74, the second stabilization loop lumen 76, the guidewire lumen 78 and the leaflet cutting device lumen 80, as shown are merely illustrative. Other arrangements, including differing relative positions of each of these lumens are contemplated. In some cases, the guidewire lumen 78 and the leaflet cutting device lumen 80 may be replaced with a single lumen (not shown) that accommodates both a guidewire and a leaflet cutting device. In some cases, the guidewire may be withdrawn before the leaflet cutting device is advanced through the elongate shaft 42. In some cases, the leaflet cutting device may be advanced over the guidewire within the elongate shaft 42.

The materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure’s scope is, of course, defined in the language in which the appended claims are expressed.

What is claimed is:

1. A medical device adapted for positioning a leaflet cutting device relative to a valve leaflet of a heart valve, the medical device comprising:
an elongate shaft extending from a proximal region to a distal region, the elongate shaft configured to accept the cutting device extending therethrough;
a stabilization loop extending out of the distal region of the elongate shaft, the stabilization loop including a first loop side terminating in a first end and a second loop side terminating in a second end; and
a control knob slidingly disposed relative to the elongate shaft and adapted to slide relative to the elongate shaft in order to translate the stabilization loop;
wherein the control knob is engaged with the stabilization loop such that rotating the control knob selectively lengthens one of the first loop side and the second loop side, thereby moving the distal region of the elongate shaft relative to the heart valve.

2. The medical device of claim 1, wherein rotating the control knob selectively lengthens one of the first loop side and the second loop side and shortens the other of the first loop side and the second loop side, thereby moving the distal region of the elongate shaft relative to the heart valve.

3. The medical device of claim 1, wherein the first end is secured to a first side of the control knob and the second end is secured to a second side of the control knob.

4. The medical device of claim 1, wherein translating the control knob distally relative to the elongate shaft causes the stabilization loop to move distally.

5. The medical device of claim 1, wherein the elongate shaft comprises a plurality of lumens.

6. The medical device of claim 5, wherein the plurality of lumens comprises a first stabilization loop lumen accommodating the first loop side extending therethrough and a second stabilization loop lumen accommodating the second loop side extending therethrough.

7. The medical device of claim 5, wherein the plurality of lumens comprises a guidewire lumen.

8. The medical device of claim 5, wherein the plurality of lumens comprises a leaflet cutting device lumen.

9. The medical device of claim 5, wherein the plurality of lumens comprise an access lumen adapted to accommodate a guidewire and/or a leaflet cutting device.

10. The medical device of claim 1, wherein the stabilization loop comprises a stainless steel wire.

11. The medical device of claim 1, wherein the stabilization loop comprises a nitinol wire.

12. A medical device adapted for positioning a leaflet cutting device relative to a valve leaflet of a heart valve, the medical device comprising:
an elongate shaft including a plurality of lumens extending from a proximal region to a distal region;
a stabilization loop extending out of the distal region of the elongate shaft, the stabilization loop including a first loop side extending through a first lumen of the plurality of lumens and a second loop side extending through a second lumen of the plurality of lumens, the first loop side terminating in a first end and the second loop side terminating in a second end; and
a control knob disposed relative to the elongate shaft and engaged with the stabilization loop such that rotating the control knob selectively lengthens one of the first loop side and the second loop side.

13. The medical device of claim 12, wherein the first end is coupled with a first side of the control knob and the second end is coupled with a second side of the control knob.

14. The medical device of claim 12, further comprising an elongate slot formed within the elongate shaft, the elongate slot allowing the control knob to translate relative to the elongate shaft, thereby causing the stabilization loop to translate relative to the elongate shaft.

15. The medical device of claim 12, wherein rotating the control knob selectively lengthens one of the first loop side and the second loop side and shortens the other of the first loop side and the second loop side, thereby moving the distal region of the elongate shaft relative to the heart valve.

16. The medical device of claim 12, wherein the plurality of lumens comprises a guidewire lumen.

17. The medical device of claim 12, wherein the plurality of lumens comprises a leaflet cutting device lumen.

18. The medical device of claim 12, wherein the stabilization loop comprises a stainless steel wire.

19. The medical device of claim 12, wherein the stabilization loop comprises a nitinol wire.

20. A medical device adapted for positioning a leaflet cutting device relative to a valve leaflet of a heart valve, the medical device comprising:
an elongate shaft defining a plurality of lumens extending through the elongate shaft, the elongate shaft extending from a proximal region to a distal region;
a stabilization loop extending out of the distal region of the elongate shaft, the stabilization loop including a first end extending proximally through a first lumen of the plurality of lumens and a second end extending proximally through a second lumen of the plurality of lumens;
a control knob slidingly disposed relative to the elongate shaft, the first end of the stabilization loop secured to a first side of the control knob and the second end of the stabilization loop secured to a second side of the control knob;
the control knob adapted to slide relative to the elongate shaft in order to translate the stabilization loop distally and proximally;
the control knob adapted to advance a first side of the stabilization loop in response to rotating the control knob a first rotational direction and to advance a second side of the stabilization loop in response to rotating the control knob a second rotational direction.
